(19) United States
(12) Patent Application Publication
(10) Pub. No.: US 2015/0071967 A1
(43) Pub. Date: Mar. 12, 2015

(54) Tolerisation-Inducing Composition

(71) Applicant: University of Bristol, Bristol (GB)

(72) Inventors: David Wraith, Bristol (GB); Elaine Hill, Bristol (GB)

(21) Appl. No.: 14/389,861

(22) PCT Filed: Apr. 2, 2013
(86) PCT No.: PCT/GB2013/050855

§ 371 Date: Oct. 1, 2014

(30) Foreign Application Priority Data
Apr. 2, 2012 (GB) 1205861.6
Mar. 14, 2013 (GB) 1304593.5

(51) Int. Cl. A61K 39/39; A61K 39/00

(52) U.S. Cl. CPC A61K 39/39; A61K 39/0008; A61K 2039/577; A61K 2039/55505; A61K 2039/55511; A61K 2039/545
USPC 424/275.1; 544/331; 548/466; 424/184.1

(57) ABSTRACT

The present invention relates to a composition comprising a tolerogenic peptide and a GSK-3 inhibitor and uses thereof. The invention also relates to the use of a GSK-3 inhibitor to accelerate the peptide-mediated shift in secretion profile of lymphocytes from pro-inflammatory to anti-inflammatory cytokines. The GSK-3 inhibitor may be used to enhance antigen-specific immunotherapy.

### FIELD OF THE INVENTION

The present invention relates to kits and compositions useful in antigen-specific immunotherapy. The kit or composition may comprise a GSK-3 inhibitor and one or more peptide(s).

### BACKGROUND TO THE INVENTION

Current treatments for allergies and autoimmune diseases rely on application of immunosuppressive drugs, the uses of which are associated with severe side-effects. There has therefore been a move towards producing antigen-specific drugs which are disease targeted and have less systemic effect than general immuno-suppression.

It has been shown to be possible to induce immunological tolerance towards particular antigens (for example autoantigens or allergens) by administration of peptide epitopes of the antigen in soluble form. Administration of soluble peptide antigens has been demonstrated as an effective means of inhibiting disease in experimental autoimmune encephalomyelitis (EAE—a model for multiple sclerosis (MS)) (Metzler and Wraith (1993) Int. Immunol. 5:1159-1165; Liu and Wraith (1995) Int. Immunol. 7:1255-1263; Anderton and Wraith (1998) Eur. J. Immunol. 28:1251-1261); and experimental models of arthritis, diabetes, and uveoretinitis (reviewed in Anderton and Wraith (1998) as above). This has also been demonstrated as a means of treating an ongoing disease in EAE (Anderton and Wraith (1998) as above).

In order for this antigen-specific immunotherapy (antigen-SIT) to be effective, peptide needs to be repeatedly administered to the patient at a high dose over an extended period.

High-dose peptide specific therapy sometimes causes a harmful immune response due to the initial burst of cell activation with subsequent proliferation and excessive cytokine release. This became evident in trials of altered peptide ligand (APL) therapy in multiple sclerosis. Treatment was terminated when it became evident that an allergic response to the peptide had been induced at the highest dose.

There is thus a need for improved antigen-specific immunotherapy methods, in which peptide therapy is effective at a lower dose, and/or for a longer time period between or after doses.

### DESCRIPTION OF THE FIGURES

FIG. 1: GSK3 inhibitors induce increased IL-10 production in Th1 cells

A. Splenocytes from a naive Tg4 mouse were cultured in the presence of peptide MBP Ac1-9 (10 μg/ml) and IL-12 with IL-2 added on day 3 of culture to polarise CD4+ cells towards a Th1 phenotype. On day 7 of culture, Th1 cells were restimulated with fresh irradiated APCs from a B10_PL mouse in the presence of DMSO (1:1000; Control), CHIR99021 (2 μM), SB216763 (5 μM) or SB627772 (10 μM) as well as peptide MBP Ac1-9 (10 μg/ml). IL-2 was added on day 3 of culture and intracellular cytokine staining for IFNγ and IL-10 carried out on day 7 of restimulation after PMA and lonomycin stimulation. Data are plots gated on live CD4⁺ cells and are representative of 5 independent experiments.

B. Quantitation of IL10⁺/IFNγ⁺ cells from 5 independent experiments as shown in A. Data are mean and SEM; *p<0.05 (ANOVA; Dunnets multiple comparison post-test).

C. Splenocytes from a naive Tg4 mouse were cultured in the presence of peptide MBP Ac1-9 (10 μg/ml) and IL-12 with IL-2 added on day 3 of culture to polarise CD4+ cells towards a Th1 phenotype. On day 7 of culture, Th1 CD4+ cells were isolated and restimulated with anti-CD3/anti-CD28 coated beads in the presence of IL-2 and DMSO (1:1000; Control), CHIR99021 (2 μM), SB216763 (5 μM) or SB627772 (10 μM). Intracellular cytokine staining for IFNγ and IL-10 was carried out on day 7 of restimulation after PMA and lonomycin stimulation. Data are plots gated on live CD4⁺ cells and are representative of 4 independent experiments.

D. Quantitation of IL10⁺/IFNγ⁺ cells from 6 independent experiments as shown in A. Data are mean and SEM; *p<0.05 (ANOVA; Dunnets multiple comparison post-test).

E. Tissue culture supernatants were taken on day 3 of restimulation and analysed for IL-10 by fluorescent bead immunoassay. Data are mean+/−SEM and are representative of 3 independent experiments.

F. Splenocytes from a naive Tg4 mouse were cultured in the presence of peptide MBP Ac1-9 (10 μg/ml) and IL-12 with IL-2 added on day 3 of culture to polarise CD4+ cells towards a Th1 phenotype. On day 7 of culture, Th1 cells were restimulated with fresh irradiated APCs from a B10_PL mouse in the presence of DMSO (1:1000; Control), CHIR99021 (2 μM), SB216763 (5 μM) or SB627772 (10□M) as well as peptide MBP Ac1-9 (10 μg/ml). IL-2 was added on day 3 of culture. On day 7 of restimulation live CD4⁺ cells were selected and stimulated with anti CD3/CD28 antibodies for 16 hrs. RNA was extracted and IL-10 mRNA quantified by qPCR. IL-10 quantities are shown relative to HPRT1 control.

G. Splenocytes from a naive Tg4 mouse were cultured in the presence of peptide MBP Ac1-9 (10 μg/ml) and IL-12 with IL-2 added on day 3 to polarise cells to a Th1 phenotype. On day 7 cells were restimulated with fresh irradiated APCs from a B10_PL mouse, peptide Ac1-9 of MBP (10 μg/ml) and the indicated concentrations of CHIR99021, SB216763 or SB627772. On day 7 of restimulation, CD4⁺ cells were lysed in RIPA buffer, subjected to Western blotting and probed with beta catenin antibody. The membrane was stripped and reprobed for GAPDH as a loading control.

H. GSK-3 inhibition does not induce IL-10 in naïve T cells.

FIG. 2: GSK3 inhibitors induce increased 11-10 production in Th2 cells

A. Splenocytes from a naive Tg4 mouse were cultured in the presence peptide MBP Ac1-9 (10 μg/ml) anti IFNγ and IL-4 with IL-2 added on day 3 of culture to polarise CD4+ cells towards a Th2 phenotype. On day 7 of culture, Th1 CD4+ cells were isolated and restimulated with anti-CD3/anti-CD28 coated beads in the presence of IL-2 and DMSO (1:1000; Control), CHIR99021 (2 μM), SB216763 (5 μM) or SB627772 (10 μM). Intracellular cytokine staining for IL-4 and IL-10 was carried out on day 7 of restimulation after PMA and lonomycin stimulation. Data are plots gated on live CD4⁺ cells and are representative of 3 independent experiments.

B. Quantitation of IL10⁺/IFNγ⁺ cells from 3 independent experiments as shown in A. Data are mean and SEM; *p<0.05 (ANOVA; Dunnets multiple comparison post-test).

C. Tissue culture supernatants were taken on day 3 of restimulation and analysed for IL-10 by fluorescent bead immunoassay. Data are mean+/−SEM and are representative of 3 independent experiments.

D. GSK-3 inhibition enhances IL-10 in Th1 cells.

FIG. 3: Exogenous IL-10 is not required for GSK3 inhibitor induced IL-10 production

A. Splenocytes from a naive Tg4 mouse were cultured in the presence of peptide MBP Ac1-9 (10 μg/ml) and IL-12 with IL-2 added on day 3 of culture to polarise CD4+ cells towards a Th1 phenotype. On day 7 of culture, Th1 cells were restimulated with fresh irradiated APCs from either a B10_PL or B10_PL IL10^{-/-} mouse in the presence of DMSO (1:1000; Control) or CHIR99021 (2 μM). IL-2 was added on day 3 of culture and intracellular cytokine staining for IFNλ and IL-10 carried out on day 7 of restimulation after PMA and lonomycin stimulation. Data are plots gated on live CD4⁺ cells and are representative of 3 independent experiments.

B. Tissue culture supernatants were taken on day 3 of restimulation and analysed for IL-10 by ELISA. Data are mean+/−SEM and are representative of 3 independent experiments.

C-E. Enhanced IL-10 production induced by GSK-3 inhibition is independent of IL-10 secretion by APC.

FIG. 4: GSK3 inhibition abrogates the ability of Th1 cells to induce EAE in an IL-10-dependent manner

Splenocytes from a naive Tg4 mouse were cultured in the presence of peptide MBP Ac1-9 (10 μg/ml) and IL-12 with IL-2 added on day 3 to polarise cells to a Th1 phenotype. On day 7 cells were restimulated with fresh irradiated APCs from a B10_PL mouse, peptide Act-9 of MBP (10 μg/ml) in the presence of DMSO (1:5000; Control) or 2 μM CHIR99021. IL-2 was added on day 3 of restimulation and on day 4 10⁷ Th1 cells were adoptively transferred to Tg4 mice by intraperitoneal injection. Recipient mice were also injected with anti IL-10R antibody or isotype control (500 μg) on the day of transfer (day 0) and on day 7. EAE was scored daily. Three similar experiments were performed.

A. Time course of disease progression. Data show the mean EAE score and SEM from each day. Each group contained 5-6 mice.

B. Disease burden in EAE days was calculated for each mouse. The median score for the Control; Isotype IgG group was significantly different from that of the CHIR99021; Isotype IgG group and the CHIR99021; Isotype IgG group was significantly different from that of the CHIR99021 anti-IL-10R group (p<0.05; Mann Whitney test).

FIG. 5: Stability of IL-10 expression and epigenetic analysis of the IL-10 promoter

A. Splenocytes from a naive Tg4 mouse were cultured in the presence of peptide MBP Ac1-9 (10 μg/ml) and IL-12 with IL-2 added on day 3 of culture to polarise CD4+ cells towards a Th1 phenotype. On day 7 of culture, Th1 cells were restimulated with fresh irradiated APCs from a B10_PL mouse in the presence of DMSO (1:1000; Control), CHIR99021 (2 μM), SB216763 (5 μM) or SB627772 (10 μM) as well as peptide MBP Ac1-9 (10 μg/ml). IL-2 was added on day 3 of culture. On day 7 of restimulation, CD4⁺ cells were restimulated with fresh irradiated APCs from a B10_PL mouse in the presence of peptide MBP Ac1-9 (10 μg/ml) only. IL-2 was added on day 3 of culture and intracellular cytokine staining for IFNλ and IL-10 carried out on day 7 of this second round of restimulation after PMA and lonomycin stimulation. Data are plots gated on live CD4⁺ cells and are representative of 2 independent experiments.

B. Schematic diagram showing the positions of primer sets used in ChIP experiments.

C, D, E. Splenocytes from a naive Tg4 mouse were cultured in the presence of peptide MBP Ac1-9 (10 μg/ml) and IL-12 with IL-2 added on day 3 of culture to polarise CD4+ cells towards a Th1 phenotype. On day 7 of culture, Th1 cells were restimulated with fresh irradiated APCs from a B10_PL mouse in the presence of DMSO (1:5000; Control) or CHIR99021 (2 μM) as well as peptide MBP Ac1-9 (10 μg/ml). IL-2 was added on day 3 of culture. Live CD4⁺ cells were selected and ChIP analysis carried out using antibodies against acetyl histone H3 (C), H3K4me3 (D) and H3K27me3 (E).

FIG. 6: GSK3 inhibition does not affect IL-10 production in naive CD4⁺ cells

A. CD4⁺/CD62L⁺ T cells were isolated from Tg4 splenocytes and cultured with anti-CD3/anti-CD28 coated beads in the presence of IL-2 and DMSO (1:1000; Control), CHIR99021 (2 μM), SB216763 (5 μM) or SB627772 (10 μM). Intracellular cytokine staining for IFNλ and IL-10 was carried out on day 7 of restimulation after PMA and lonomycin stimulation. Data are plots gated on live CD4⁺ cells and are representative of 3 independent experiments.

B. Tissue culture supernatants were taken on day 3 of restimulation and analysed for IL-10 by fluorescent bead immunoassay. Data are mean+/−SEM of 3 independent experiments.

FIG. 7: Exogenous IL-10 is not required for GSK3 inhibitor induced IL-10 production in Th2 cells.

FIG. 8: GSK3 inhibitor treatment increases IL-10 secretion in vivo. Data are plots gated on live CD4⁺ cells and the % IL-10 expressing cells shown graphically. *p<0.05

FIG. 9: GSK-3 inhibition suppresses the encephalitogenic properties of Th1 cells.

FIG. 10: Differential effects of GSK-3 inhibition on IL-10 induction versus β-catenin turnover in lymphocytes.

### SUMMARY OF ASPECTS OF THE INVENTION

The present inventor has shown that GSK-3 inhibition enhances tolerance induction by peptides.

GSK-3 inhibitors may therefore be used as “tolerogenic adjuvants” to enhance antigen-specific immunotherapy.

Thus, in a first aspect, the present invention provides a composition which comprises a peptide and a glycogen synthase kinase-3 (GSK-3) inhibitor.

The peptide may be a tolerogenic peptide.

The peptide may be an antigenic peptide. For example the peptide may be from a self-antigen or an allergen.

The GSK-3 inhibitor may comprise a lithium ion, CHIR99021, SB216763 or SB627772.

In a second aspect the present invention provides a composition according to the first aspect of the invention for use in a method for treating and/or preventing an autoimmune disease, an allergic reaction, a condition associated with transplant rejection, or a condition in which inflammation and CD4+ T cells contribute to pathogenesis such as cerebral malaria, atherosclerosis, type II diabetes and neuropathic pain.

In a third aspect, the present invention provides a kit comprising a peptide and a GSK-3 inhibitor for separate, simultaneous or sequential administration.

The peptide maybe a tolerogenic peptide.

In a fourth aspect, the present invention provides a method for treating and/or preventing a disease associated with pro-inflammatory T cells which comprises the step of administering a peptide, such as a tolerogenic peptide, and a GSK-3 inhibitor to the subject.

The peptide and GSK-3 inhibitor may be administered simultaneously, sequentially or separately.

The method may comprise the following two treatment stages:
(i) an initial treatment stage with peptide and GSK-3 inhibitor; and
(ii) a subsequent treatment phase with peptide in the absence of GSK-3 inhibitor

The disease associated with pro-inflammatory T cells may be selected from: an autoimmune disease, an allergic reaction, a condition associated with transplant rejection, and a condition in which inflammation and CD4+ T cells contribute to pathogenesis such as cerebral malaria, atherosclerosis, type II diabetes and neuropathic pain.

The subject may have a pre-existing condition, or may be about to undergo or undergoing transplantation.

The subject may have a pre-existing Th1/Th17 or Th2 immune response specific for a self-antigen or allergen.

In a fifth aspect, the present invention provides a GSK-3 inhibitor for use in enhancing antigen-specific immunotherapy.

In a sixth aspect, the present invention provides GSK-3 inhibitor for use in accelerating the peptide-mediated shift in secretion profile of T cells from pro-inflammatory to anti-inflammatory cytokines.

In a seventh aspect, the present invention provides a composition according to the first aspect of the invention or a kit according to the third aspect of the invention for use in treating and/or preventing a disease associated with pro-inflammatory T cells.

In an eighth aspect, the present invention provides the use of a tolerogenic peptide and GSK-3 inhibitor in the manufacture of a medicament for use in treating and/or preventing a disease associated with pro-inflammatory T cells.

The disease associated with proinflammatory T cells may be selected from: an autoimmune disease, an allergic reaction, a condition associated with transplant rejection, and a condition in which inflammation and CD4+ T cells contribute to pathogenesis such as cerebral malaria, atherosclerosis, type II diabetes and neuropathic pain.

### DETAILED DESCRIPTION

### GSK-3 Inhibitor

Glycogen synthase kinase-3 (GSK-3) was initially identified as an enzyme involved in glycogen metabolism. In recent years it has been shown to have key roles in regulating a diverse range of cellular functions. GSK-3 has numerous physiological substrates which include transcription factors as well as enzymes involved in regulating metabolism.

Several potent GSK-3 inhibitors have been developed over the last 15 year for the treatment of diabetes and neurodegenerative diseases, such as Alzheimer's disease.

GSK-3 is inhibited in response to a variety of agonists as a result of the phosphorylation of a single serine residue (Ser21 in the α-isoform and Ser-9 in the β-isoform). GSK-3 may therefore be inhibited by various protein kinases, including protein kinase B (also known as AKT), S6 kinase and ribosomal protein S6 kinase.

The GSK-3 inhibitor may be a small molecule inhibitor, such as a malemide. The inhibitor may be selected from the following group: SB216763, SB415286, CHIR98014, CHIR99021, CHIR98023, AR A014418, 1-Azakenpaullone and Bis-7-indoylmaleimide. The chemical structure of these inhibitors in given in Cohen and Goedert (2004) (Nat. Rev. Drug Disc. 3:479-487, see FIG. 4). The GSK-3 inhibitor may comprise a lithium ion or a thienyl or phenyl α-halomethylketone.

There is a possibility that some GSK-3 inhibitors may be oncogenic. Many components of the Wnt signalling pathway are overexpressed or mutated in several types of cancer. For example, about 15% of colon cancers arise from an initiating mutation in β-catenin. GSK-3 inhibitors might be expected to mimic the Wnt signalling pathway and therefore be potentially oncogenic. Some GSK-3 inhibitors such as SB415286 and lithium ions have all been shown to elevate the level of 13-catenin. However, GSK-3 inhibitors, such as lithium ions, have been used as mood stabilisers for more than 50 years and their use is not known to be associated with an increased risk of cancer.

The present inventors have shown that CHIR99021 and SB216763 differ in their effect on β-catenin levels (see Example 5). In the presence of CHIR99021 the degradation of β-catenin appears to be inhibited, leading to build up of the β-catenin protein. The presence of SB216763, on the other hand, does not appear to cause a significant effect on β-catenin levels. For safety reasons, a GSK-3 inhibitor may be chosen which does not significantly increase the level of β-catenin in cells at a concentration that clearly increases IL-10 levels in lymphocytes.

### Peptide

The term “peptide” is used in the normal sense to mean a series of residues, typically L-amino acids, connected one to the other typically by peptide bonds between the α-amino and carboxyl groups of adjacent amino acids The term includes modified peptides and synthetic peptide analogues.

Peptides that bind to MHC class I molecules are typically 7 to 13, more usually 8 to 10 amino acids in length. The binding of the peptide is stabilised at its two ends by contacts between atoms in the main chain of the peptide and invariant sites in the peptide-binding groove of all MHC class I molecules. There are invariant sites at both ends of the groove which bind the amino and carboxy termini of the peptide. Variations in peptide length are accommodated by a kinking in the peptide backbone, often at proline or glycine residues that allow the required flexibility.

Peptides which bind to MHC class II molecules are typically between 8 and 20 amino acids in length, more usually between 10 and 17 amino acids in length, and can be much longer. These peptides lie in an extended conformation along the MHC II peptide-binding groove which (unlike the MHC class I peptide-binding groove) is open at both ends. The peptide is held in place mainly by main-chain atom contacts with conserved residues that line the peptide-binding groove.

Peptides may be made using chemical methods. For example, peptides can be synthesized by solid phase techniques, cleaved from the resin, and purified by preparative high performance liquid chromatography. Automated synthesis may be achieved, for example, using the ABI 43 1 A Peptide Synthesizer (Perkin Elmer) in accordance with the instructions provided by the manufacturer.

The peptide may alternatively be made by recombinant means, or by cleavage from a longer polypeptide. For example, the peptide may be obtained by cleavage from the target antigen. The composition of a peptide may be confirmed by amino acid analysis or sequencing (e.g., the Edman degradation procedure).

The peptide may be derivable from a target antigen. A target antigen is a molecule (for example a protein or glycoprotein) which is processed by antigen presenting cells (APC) and recognised by T cells during the course of the disease. The target antigen will, of course, depend on the target disease. The peptide may be derivable from a fragment of the antigen which arises by natural processing of the antigen by an APC. The target antigen may be an allergen or an autoantigen. The peptide may comprise a T cell epitope.

The peptide may be “tolerogenic” in the sense that it is capable of inducing tolerance to an antigen when administered in a soluble form in vivo. The peptide may be useful in antigen-specific immunotherapy (antigen-SIT).

A “tolerogenic peptide” may be capable of inducing tolerance (to some extent) alone, or it may only be capable of inducing appreciable levels of tolerance in combination with a GSK-3 inhibitor.

The peptide is capable of binding to an MHC molecule. The peptide is capable of binding to an MHC molecule and being presented to a T cell.

Tolerogenic peptides are capable of binding to an MHC molecule and being presented to a T cell without the need for antigen processing. They can bind to MHC in a processing-free presentation system, such as fixed antigen presenting cells or plate-bound MHC, and be presented to a T cell.

The peptide may be useful in the treatment and/or prevention of a disease. In particular, the peptide may be useful in the treatment and/or prevention of a disease which is mediated by autoreactive T cells. Hypersensitivity reactions are particularly amenable to treatment/prevention using the peptide of the present invention, for example allergy, autoimmunity and transplant rejection.

### Antigen-Specific Immunotherapy

Antigen-specific Immunotherapy (antigen-SIT) is the induction of tolerance to an antigen by the administration of one or more peptide epitopes of that antigen in soluble form.

As used herein, the term “tolerogenic” means capable of inducing tolerance.

Tolerance is the failure to respond to an antigen. Tolerance to self-antigens is an essential feature of the immune system: when this is lost, autoimmune disease can result. The adaptive immune system must maintain the capacity to respond to an enormous variety of infectious agents while avoiding autoimmune attack of the self-antigens contained within its own tissues. This is controlled to a large extent by the sensitivity of immature T lymphocytes to apoptotic cell death in the thymus (central tolerance). However, not all self-antigens are detected in the thymus, so death of self-reactive thymocytes remains incomplete. There are thus also mechanisms by which tolerance may be acquired by mature self-reactive T lymphocytes in the peripheral tissues (peripheral tolerance).

Tolerance may result from or be characterised by the induction of anergy in at least a portion of CD4+ T cells. In order to activate a T cell, a peptide must associate with a “professional” APC capable of delivering two signals to T cells. The first signal (signal 1) is delivered by the MHC-peptide complex on the cell surface of the APC and is received by the T cell via the TCR. The second signal (signal 2) is delivered by costimulatory molecules on the surface of the APC, such as CD80 and CD86, and received by CD28 on the surface of the T cell. It is thought that when a T cell receives signal 1 in the absence of signal 2, it is not activated and, in fact, becomes anergic. Anergic T cells are refractory to subsequent antigenic challenge, and may be capable of suppressing other immune responses. Anergic T cells are thought to be involved in mediating T cell tolerance.

It has been shown that, when tolerance is induced by peptide administration, the capacity of antigen-specific CD4+ T cells to proliferate is reduced. Also, the production of IL-2, IFN-γ and IL-4 production by these cells is down-regulated, but production of IL-10 is increased. Neutralisation of IL-10 in mice in a state of peptide-induced tolerance has been shown to restore susceptibility to disease completely. It has been proposed that a population of regulatory cells persist in the tolerant state which produce IL-10 and mediate immune regulation (Burkhart et al (1999) Int. Immunol. 11:1625-1634).

The induction of tolerance can therefore be monitored by various techniques including:
(a) reduced susceptibility to contract the disease for which the peptide is a target epitope in vivo;
(b) the induction of anergy in CD4+ T cells (which can be detected by subsequent challenge with antigen in vitro);
(c) changes in the CD4+ T cell population, including reduction in proliferation;
(ii) down-regulation in the production of IL-2, IFN-γ and IL-4; and
(iii) increase in the production of IL-10.

Antigen-specific immunotherapy (SIT) has used for the treatment of allergies and autoimmune diseases.

Subcutaneous or oral/sublingual administration of allergens has been used for the successful treatment of a wide range of allergies including those to bee venom, cow's milk, peanut and birch pollen.

There has also been some success using antigen-SIT to treat autoimmune diseases such as multiple sclerosis, rheumatoid arthritis, systemic lupus erythematosus and type I diabetes.

### Composition

The first aspect of the invention relates to a composition comprising a GSK-3 inhibitor and at least one peptide, for example at least one tolerogenic peptide.

The peptide may be useful for antigen-SIT. The GSK-3 inhibitor may act as a “tolerogenic adjuvant”. The tolergenic adjuvant is thus used alongside antigen-specific immunotherapy as an adjunctive. Adjunctive therapy is defined as an additional substance, treatment, or procedure used for increasing the efficacy or safety of the primary substance, treatment, or procedure or for facilitating its performance.

In order to treat or prevent a disease, it may be necessary to target a number of different T cell clones in order to induce tolerance effectively. Hence a plurality of peptides may be administered to an individual in order to prevent or treat a disease.

The composition may therefore comprise one or a plurality of peptides.

The composition may, for example, comprise between 2 and 50 peptides, preferably between 2 and 15 peptides. The peptides may be derivable from the same or different target antigen(s).

The composition may be in the form of a kit, in which some or each of the peptides are provided separately for simultaneous, separate or sequential administration.

Alternatively (or in addition) if the composition (or any part thereof) is to be administered in multiple doses, each dose may be packaged separately.

The composition may comprise a therapeutically or prophylactically effective amount of one or more peptides and optionally a pharmaceutically acceptable carrier, diluent or excipient.

Also, in the compositions of the present invention, one or more peptides may be admixed with any suitable binder(s), lubricant(s), suspending agent(s), coating agent(s), or solubilising agent(s).

### Kit

In a third aspect, the present invention provides a kit comprising a peptide and a GSK-3 inhibitor for separate, simultaneous or sequential administration.

The peptide(s) and GSK-3 inhibitor(s) are as defined in relation to the composition of the first aspect of the invention. The peptide(s) and GSK-3 inhibitor(s) may be mixed before administration, or they may be administered separately. The peptide(s) and GSK-3 inhibitor(s) may be administered by the same or different administration routes (see below).

The kit may comprise one or more peptides. Where the kit comprises a plurality of peptides, they may be mixed, in the form of a peptide “cocktail” or they may be packaged separately for separate, simultaneous or sequential administration.

The kit may also comprise instructions for use.

### Administration

The composition of the invention may be administered by any route suitable for antigen-specific immunotherapy. For example, the composition may be administered by a mucosal route.

The composition may be administered subcutaneously (s.c.), intraperitoneally (i.p.), intravenously (i.v.) or intranasally (i.n.) or by an oral/sublingual route.

Where the peptide(s) and GSK-3 inhibitor are in the form of a kit, they may be administered by the same or different administration route, at the same or different times.

### Method of Treatment

In a fourth aspect, the present invention provides a method for treating and/or preventing a disease associated with pro-inflammatory T cells which comprises the step of administering a peptide and a GSK-3 inhibitor to the subject.

The peptide and GSK-3 inhibitor may be administered simultaneously, sequentially or separately.

The method may comprise the following two treatment stages:
(i) an initial treatment stage with peptide and GSK-3 inhibitor; and
(ii) a subsequent treatment phase with peptide in the absence of GSK-3 inhibitor

The broad substrate specificity of GSK-3 suggests that long-term administration of such inhibitors could potentially lead to complications. The two-step treatment method, however, should avoid any such problem. People suffering from autoimmune disease or allergy will have pre-existing Th1/Th17 or Th2 immune responses specific for self-antigens or allergens; short-term treatment with antigenic peptide and GSK-3 inhibitor will shift the cytokine production of the cells hence creating an anti-inflammatory response focused on the self-antigen or allergen; continued treatment with antigenic peptide alone should be sufficient to maintain the antigen-specific, anti-inflammatory phenotype. This avoids the need for continuous administration of the GSK-3 inhibitor.

### Disease/Condition

The disease or condition may be mediated by CD4+ T cell responses. For example, the disease may be established or maintained by an inappropriate or excessive CD4+ T cell response.

The composition or kit may be useful in the treatment of hypersensitivity disorders. Hypersensitivity reactions include:
(i) allergies, resulting from inappropriate responses to innocuous foreign substances;
(ii) autoimmune diseases, resulting from responses to self tissue antigens; and
(iii) graft rejection, resulting from responses to a transplant.

Examples of allergies include, but are not limited to: hay fever, extrinsic asthma, insect bite and sting allergies, food and drug allergies, allergic rhinitis, bronchial asthma, chronic bronchitis, anaphylactic syndrome, urticaria, angioedema, atopic dermatitis, allergic contact dermatitis, erythema nodosum, erythema multiforme, Stevens-Johnson Syndrome, rhinoconjunctivitis, conjunctivitis, cutaneous necrotizing venulitis, inflammatory lung disease and bullous skin diseases.

Examples of the autoimmune diseases include, but are not limited to: rheumatoid arthritis (RA), myasthenia gravis (MG), multiple sclerosis (MS), systemic lupus erythematosus (SLE), autoimmune thyroiditis (Hashimoto's thyroiditis), Graves' disease, inflammatory bowel disease, autoimmune uveoretinitis, polymyositis and certain types of diabetes, systemic vasculitis, polymyositis-dermatomyositis, systemic sclerosis (scleroderma), Sjogren's Syndrome, ankylosing spondylitis and related spondyloarthropathies, rheumatic fever, hypersensitivity pneumonitis, allergic bronchopulmonary aspergillosis, inorganic dust pneumoconioses, sarcoidosis, autoimmune hemolytic anemia, immunological platelet disorders, cryopathies such as cryofibrinogenemia and autoimmune polyendocrinopathies.

A variety of tissues are commonly transplanted in clinical medicine, including kidney, liver, heart lung, skin, cornea and bone marrow. All grafts except corneal and some bone marrow grafts usually require long-term immunosuppression at present.

The composition or kit may be for use in the treatment and/or prevention of diabetes, in which case the peptide(s) may be derivable from the target antigen IA2.

The composition or kit may be for use in the treatment and/or prevention of multiple sclerosis (MS). Multiple sclerosis (MS) is a chronic inflammatory disease characterised by multiple demyelinating lesions disseminated throughout the CNS white matter and occurring at various sites and times. MS is thought to be mediated by autoreactive T cells. The peptide(s) may be derivable from one of the autoantigens associated with MS, in particular myelin basic protein (MBP) or proteolipid protein (PLP).

In transplant rejection the GSK-3 inhibitor may be given alone or in conjunction with an ‘indirectly presented MHC epitope’ i.e. one or more peptides derived from the donor MHC alloantigen.

In cerebral malaria, the GSK-3 inhibitor may be given with one or more malaria T cell epitope(s) from, for example, the circumsporozoite protein or thrombospondin-related adhesion protein.

In atherosclerosis, the GSK-3 inhibitor may be given with one or more T cell epitope(s) from, for example, oxidised LDL or heat shock protein.

In neuropathic pain, the GSK-3 inhibitor may be given with one or more peripheral nerve T cell epitope(s) from, for example, the P0 or P2 antigen.

### Cytokine Secretion Profile

In a sixth aspect, the present invention provides a GSK-3 inhibitor for use in accelerating the peptide-mediated shift in secretion profile of T cells from pro-inflammatory to anti-inflammatory cytokines.

The T cells may be CD4+ T cells.

Antigen-specific immunotherapy works by increasing the expression of IL-10. Interleukin-10 is itself an anti-inflammatory but also has a knock on anti-inflammatory effect on dendritic cells. Interleukin-10 down regulates IL-12 in dendritic cells preventing Th1 cell differentiation.

Thus, in antigen-SIT, peptide(s) cause a shift in secretion profile of T cells towards an IL-10 secreting suppressive T cell phenotype.

Pro-inflammatory Th1 cells produce IL-2 and IFNγ, whereas pro-inflammatory Th2 cells produce IL-4. Thus a “pro-inflammatory T cell cytokine secretion profile” may be characterised by the secretion of IL-2, IFNγ, and/or IL-4.

CD4+ T cells with regulatory function (T reg cells) secrete IL-10. Thus an “anti-inflammatory T cell cytokine secretion profile” is characterized by the secretion of IL-10.

The present inventors have found that GSK-3 inhibitors are capable of accelerating the peptide-mediated shift in the enhanced IL-10 profile of T cells associated with antigen-SIT. They can thus be used as an adjunctive therapy with antigen-SIT.

Without wishing to be bound by theory, the present inventors believe that this is because GSK-3 is involved in the switch mechanism between pro- and anti-inflammatory cytokine production. Phosphorylation of CBP by GSK-3 causes CBP to be recruited to the NFKB binding site in the promoter sequence of pro-inflammatory cytokines such as IL-2. This therefore causes an up regulation in pro-inflammatory cytokines. Inhibition of GSK-3 prevents this phosphorylation, allowing CBP to bind to the CREB binding sites in the IL-10 promoter sequence, upregulating IL-10 production. This interaction between GSK-3 and CBP is therefore a switch mechanism by which CD4+ T cells alter their expression profiles between pro-inflammatory and anti-inflammatory.

The invention will now be further described by way of Examples, which are meant to serve to assist one of ordinary skill in the art in carrying out the invention and are not intended in any way to limit the scope of the invention.

### EXAMPLES

### Example 1

### GSK-3 Inhibition does not Induce IL-10 in NaïVe T Cells

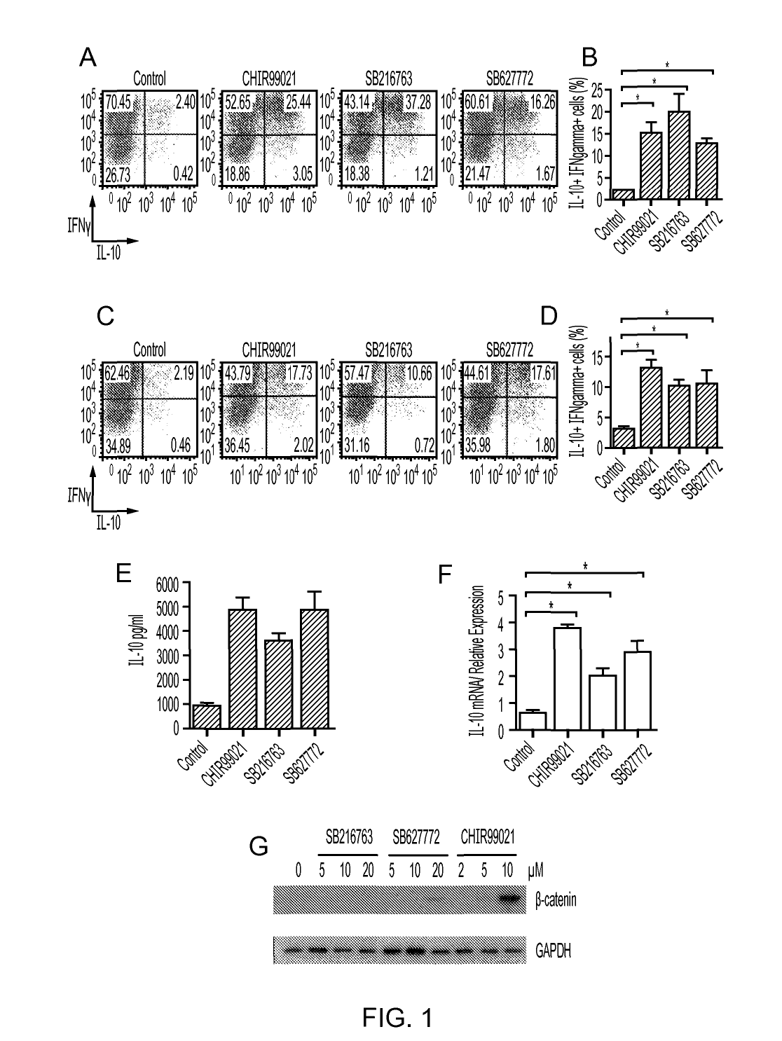

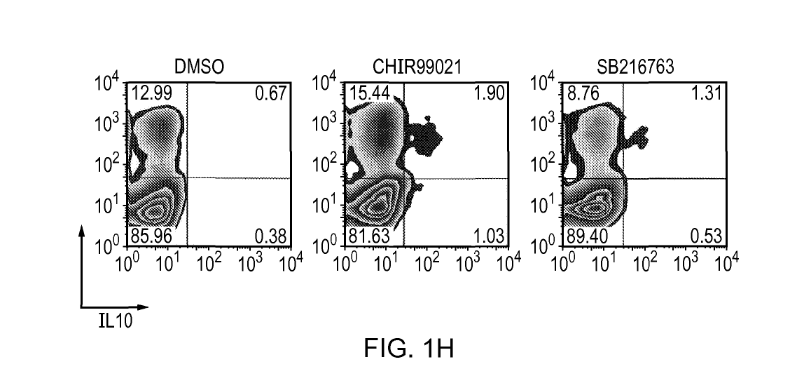

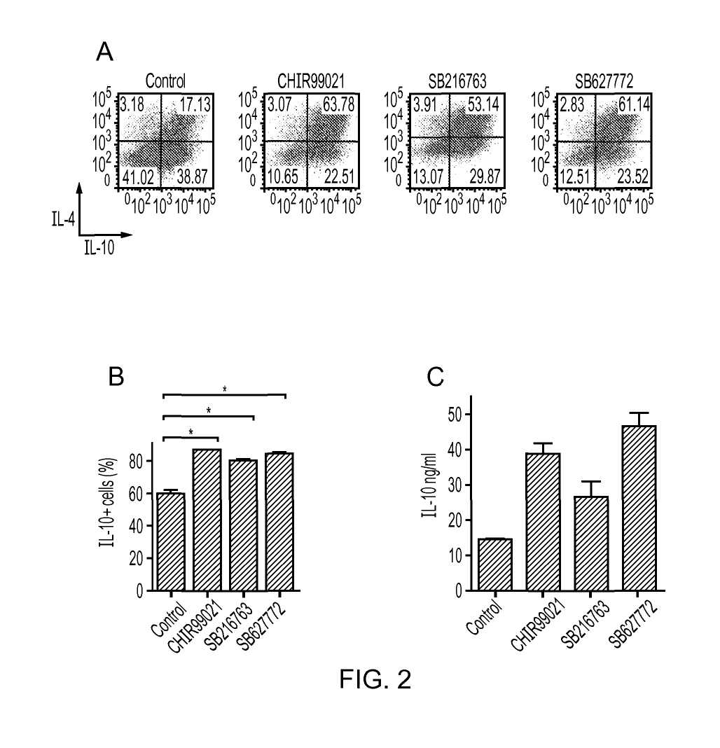

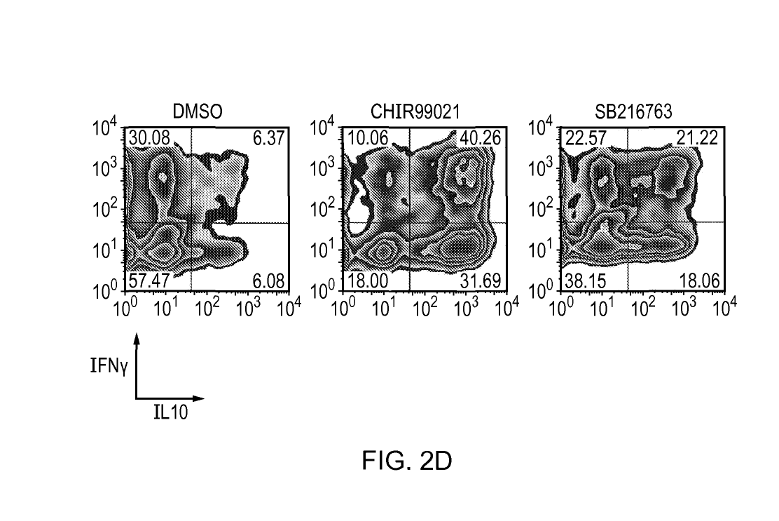

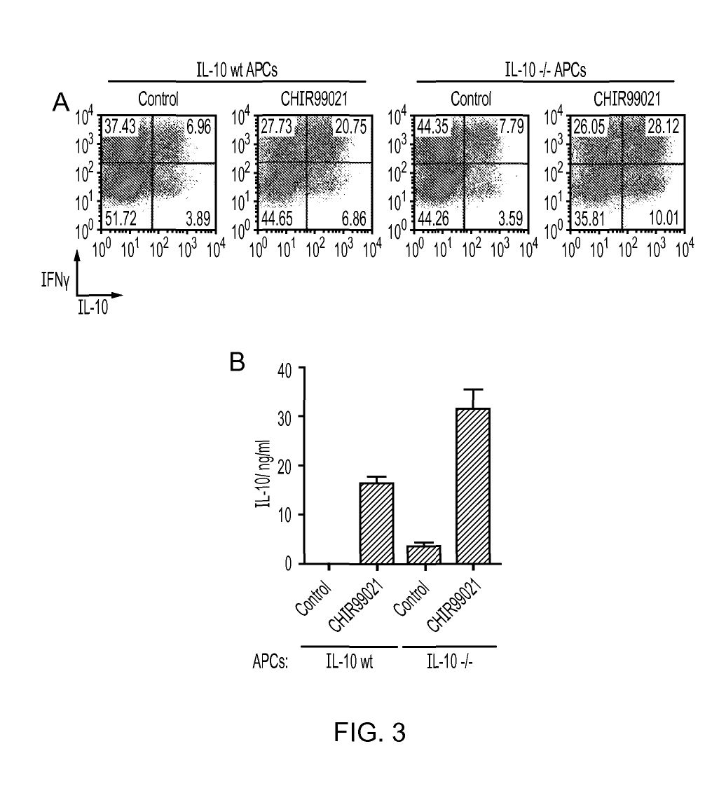

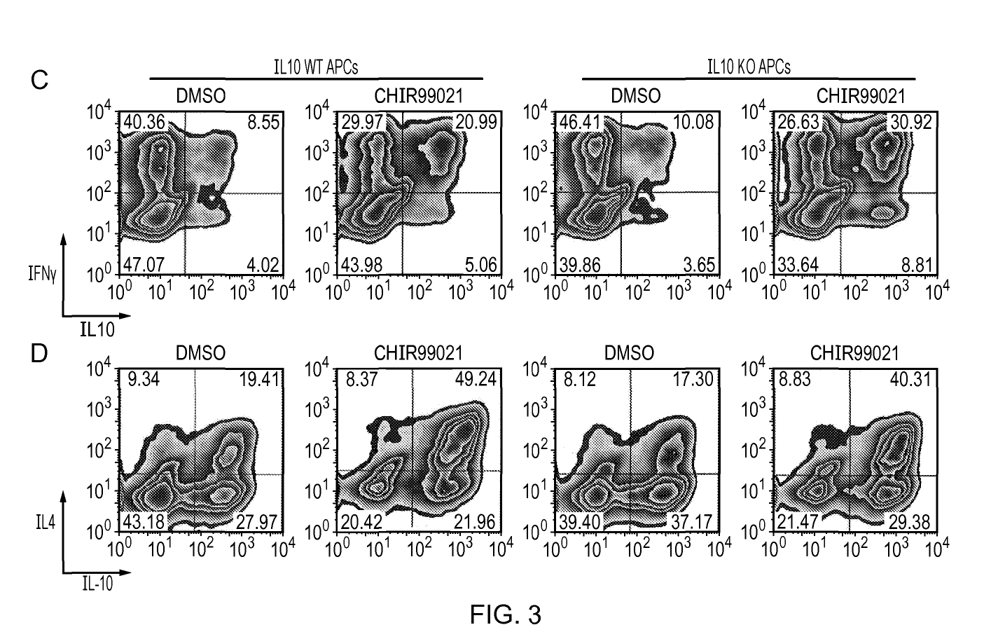

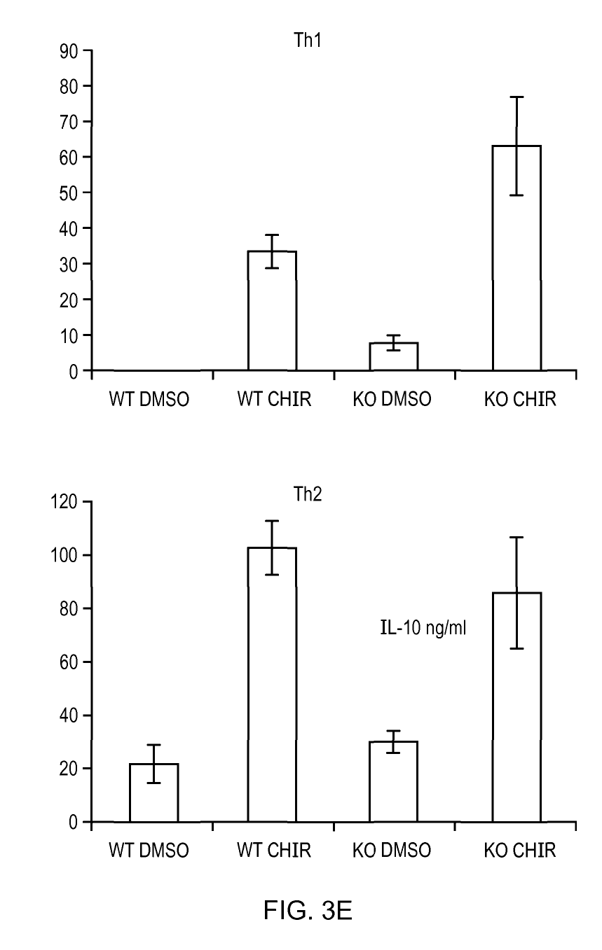

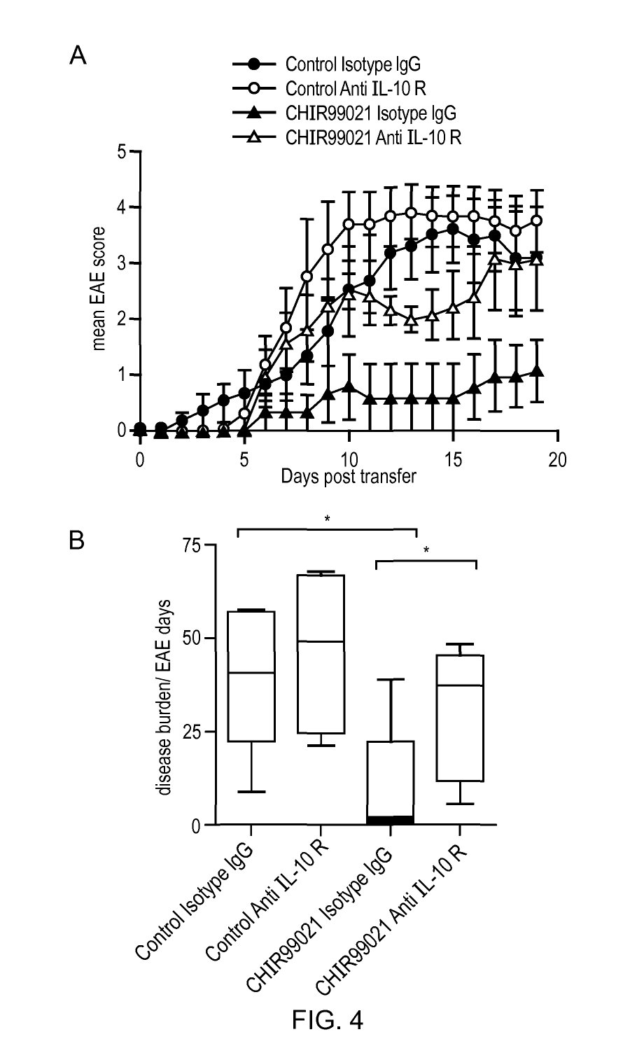

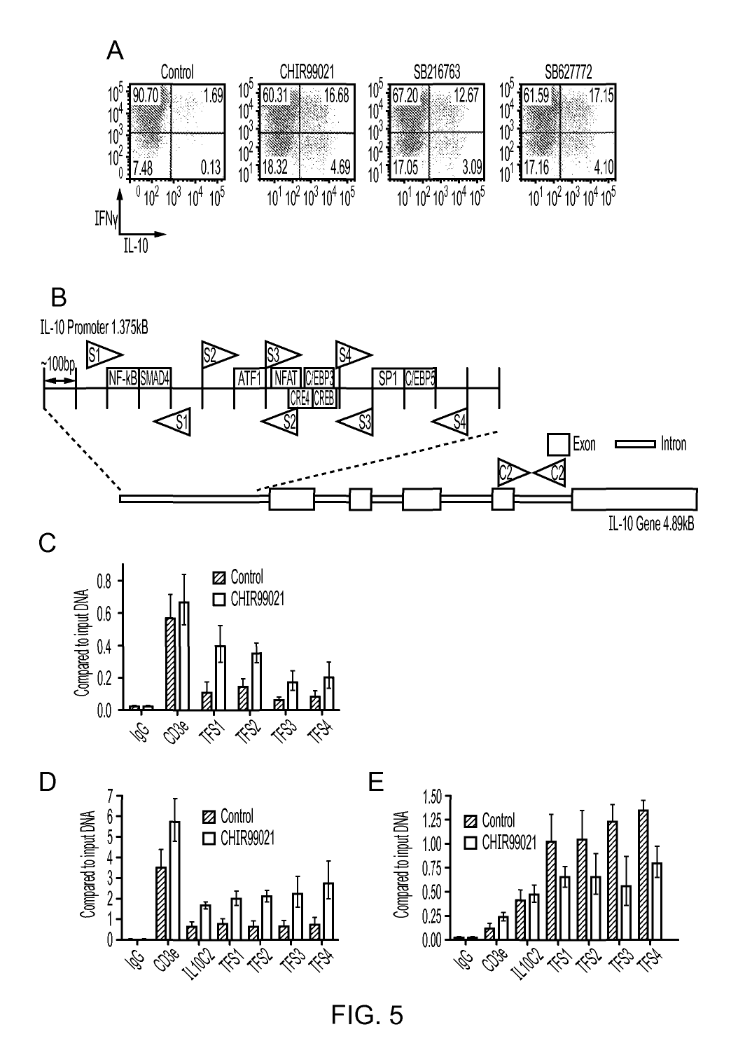

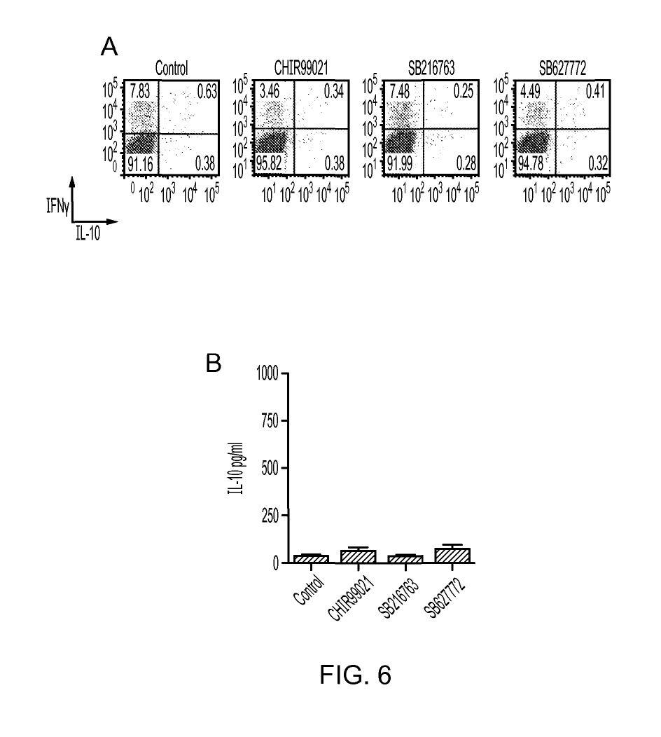

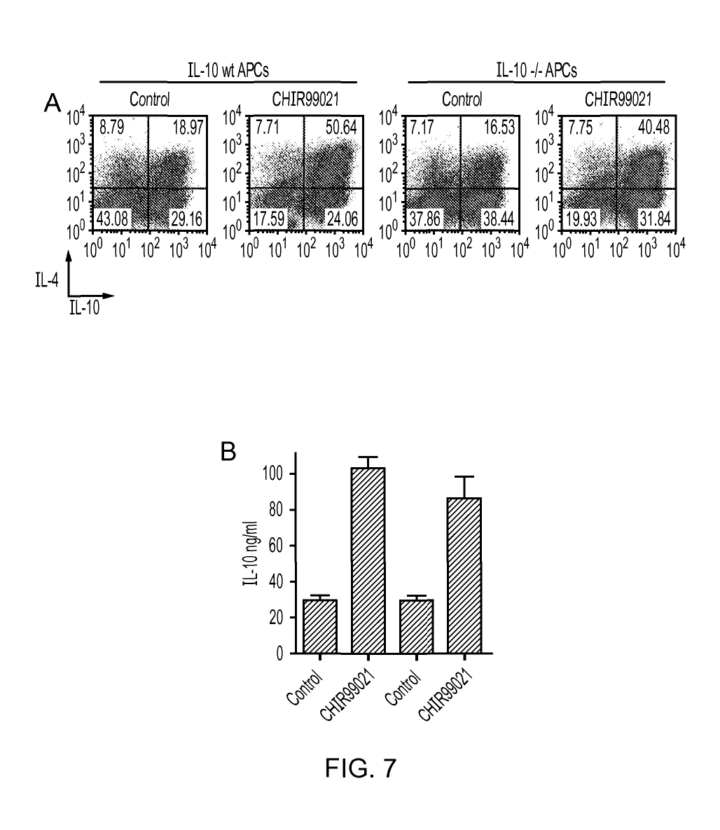

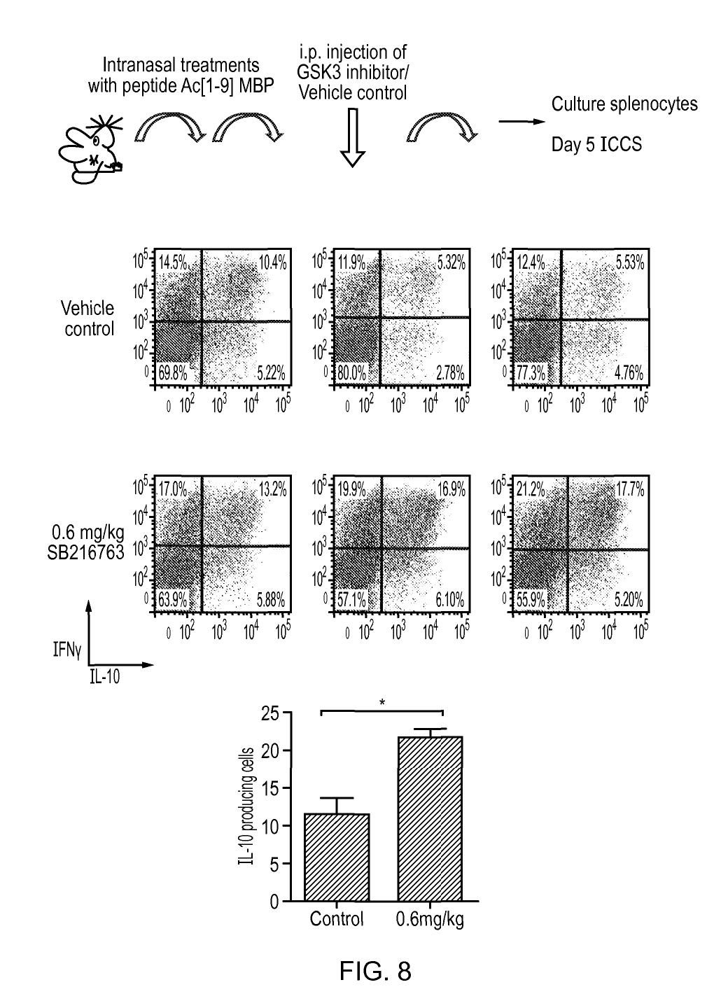

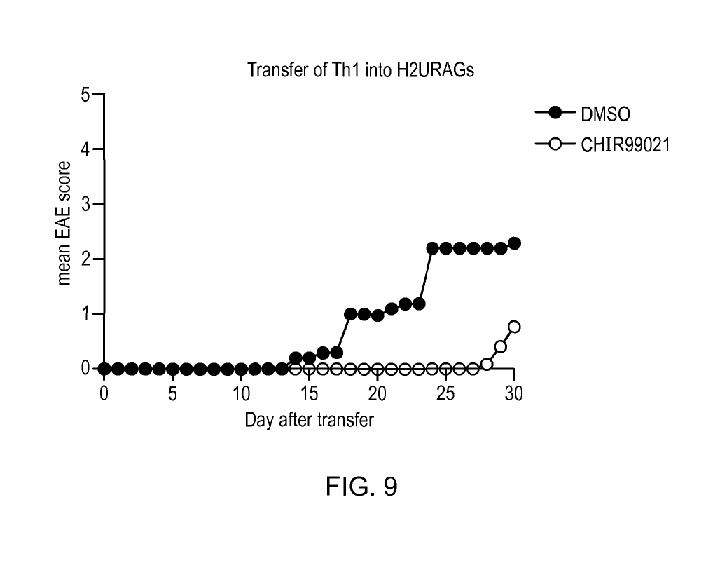

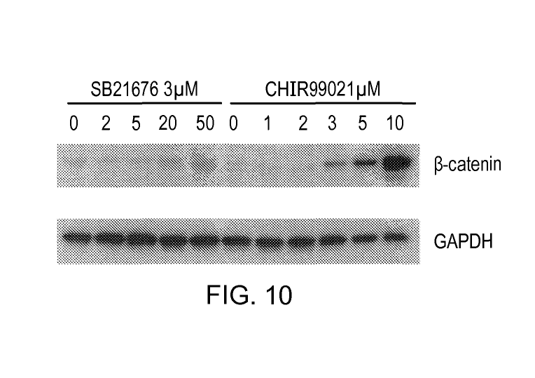

CD4+ cells purified from a naive Tg4 spleen were cultured in the presence of 2 μM CHIR99021, 3 μM SB216763 or 0.02% DMSO (vehicle control) with anti CD3/anti CD28 Dynabeads. Cells were analysed by ICCS on Day 7 after stimulation. The results are shown in FIG. 1H. Intracellular IL-10 levels were not significantly affected by treatment with either GSK-3 inhibitor (CHIR99021 or SB216763).

### Example 2

### GSK-3 Inhibition Enhances IL-10 in Th1 and Th2 Cells

Splenocytes from a naive Tg4 mouse were cultured in the presence of peptide MBP Ac1-9 (10 μg/ml) and IL12 with IL2 added on day 3 to polarise cells to a Th1 phenotype. On day 7, cells were restimulated with anti CD3/anti CD28 Dynabeads and 2 μM CHIR99021, 3 μM SB216763 or 0.02% DMSO (vehicle control). Cells were analysed by ICCS on Day 7 after stimulation. The results are shown in FIG. 2D. Intracellular IL-10 levels were increased following treatment with either GSK-3 inhibitor (CHIR99021 or SB216763).

In a separate experiment, CD4+ cells from Tg4 mice which express TCR specific for the peptide Ac[1-9] of MBP were polarised to a Th1 phenotype and then cultured in the presence of GSK3 inhibitors. Three structurally distinct inhibitors, CHIR99021, SB216763 and SB627772 were used to minimise effects of off-target inhibition. IL-10 protein was found to be significantly increased in cells treated with GSK3 inhibitors in cultures of CD4⁺ cells restimulated with APCs and peptide as well as those restimulated with anti-CD3/anti-CD28 coated beads (FIG. 1A-E), showing that GSK3 inhibitors act directly on the CD4⁺ cells. The upregulation was shown to be at the mRNA level by qPCR analysis (FIG. 1F). Naive CD4⁺CD62L⁺ T-cells purified from spleens of Tg4 mice and cultured with peptide Ac[1-9] of MBP did not show any change in IL-10 production when cultured in the presence of GSK3 inhibitors (FIG. 6).

Th2 cells also increased IL-10 production in response to GSK3 inhibition (FIG. 2). As well as an increase in the overall number of cells producing IL-10 there was a particularly large increase in cells expressing both IL-10 and IL-4.

### Example 3

### Enhanced IL-10 Production Induced by GSK-3 Inhibition is Independent of IL-10 Secretion by APC

Splenocytes from a naive Tg4 mouse were cultured in the presence of either: (a) peptide MBP Ac1-9 (10 μg/ml) and IL12 with IL2 added on day 3 to polarise cells to a Th1 phenotype; or (b) IL4 and anti IFNγ with IL2 added on day 3 to polarise cells to a Th2 phenotype. On day 7 CD4+ cells were restimulated with fresh irradiated APCs from either WT or IL10 knockout mice, 2 μM CHIR99021 or 0.02% DMSO (vehicle control) and peptide Act-9 of MBP (10 μg/ml). The results are shown in FIG. 3 (C) and (D). Supernatants were taken in triplicate on day 3 and analysed for IL-10 by ELISA (FIG. 3 (E)). Neither the intracellular level of IL-10 in the APC nor the production of IL-10 by the APC was enhanced during the presentation assay, ruling out the possibility that the enhanced IL-10 observed in Th1 cells in Example 2 was due to enhance IL-10 production by the APC.

In a separate experiment, Th1 CD4+ cells from Tg4 mice were cultured with APCs from IL-10 mice in the presence of GSK3 inhibitors (FIGS. 3A and B). It was found that IL-10 from APCs was not necessary for the increased production of IL-10 from CD4⁺ induced by GSK3 inhibition, in fact exogenous IL-10 appeared to partially suppress IL-10 secretion from CD4⁺ cells.

### Example 4

### GSK-3 Inhibition Suppresses the Encephalitogenic Properties of Th1 Cells

Tg4 splenocytes were stimulated with Ac1-9 peptide+IL-12 for 7 days. Th1 cells were restimulated with peptide±GSK-3 inhibitor for 3 days. 5×10⁶ cells were collected and transferred ip/iv into RAG deficient H-2^{u} recipient mice and mice were graded for EAE. The results are shown in FIG. 9. Th1 cells restimulated with peptide together with a GSK-3 inhibitor cause greater suppression of EAE that Th1 cells restimulated with peptide alone. GSK-3 inhibition promotes the efficacy of peptide therapy by accelerating conversion of cells to the IL-10 phenotype.

In a separate experiment, CD4+ cells from Tg4 mice were polarised to a Th1 phenotype, cultured with GSK3 inhibitor or vehicle control and adoptively transferred to Tg4 mice to induce EAE. Mice also received anti-IL-10R to neutralise IL-10 or control isotype IgG. Culture of Th1 cells with GSK3 inhibitor resulted in a significant decrease in disease burden in mice treated with control isotype IgG (FIG. 4 B). The decrease in EAE scores in this group was sustained for the entire disease course (FIG. 4A). Mice receiving GSK3 inhibitor treated cells and anti IL-10R antibody however showed a significant increase in disease burden compared with those receiving the control isotype antibody (FIG. 4 B). This shows that the decrease in pathogenicity resulting from GSK3 inhibitor treatment is dependent on the action of IL-10.

### Example 5

### Differential Effects of GSK-3 Inhibition on IL-10 Induction Versus β-Catenin Turnover in Lymphocytes

Splenocytes from a naive Tg4 mouse were cultured in the presence of peptide MBP Ac1-9 (10 μg/ml) and IL12 with IL2 added on day 3 to polarise cells to a Th1 phenotype. On day 7 cells were restimulated with fresh irradiated APCs, peptide Ac1-9 of MBP (10 μg/ml) and the indicated concentrations of SB216763 or CHIR99021. On day 7 of restimulation, cells were lysed in RIPA buffer, subjected to Western blotting and probed with beta catenin antibody. The membrane was stripped and reprobed for GAPDH as a loading control. The results are shown in FIG. 10. GSK-3 consititutively phosphorylates the β-catenin protein, leading to its degradation. In the presence of CHIR99021, the degradation of β-catenin appears to be inhibited, leading to build up of the β-catenin protein. SB216763, on the other hand, does not appear to cause a significant effect on β-catenin levels.

In a separate experiment, Western blot analysis of CD4⁺ cells showed no accumulation of β-catenin when CD4⁺ cells were cultured with GSK3 inhibitors at concentrations leading to increased IL-10 (2 μM CHIR99021, 5 μM SB216763, 10 μM SB627772) (FIG. 1G).

### Example 6

### The IL-10 Promoter Undergoes Epigenetic Changes in Th1 Cells when GSK3 is Inhibited

The Th1 cells used to induce EAE by adoptive transfer had been cultured with inhibitor/vehicle control and subsequently washed several times in order to remove all exogenous inhibitor before transfer. The IL-10 mediated suppression of EAE by the inhibitor treatment was sustained throughout the course of the experiment, suggesting that the cellular changes effected by the inhibitor were long-lasting and heritable throughout several rounds of proliferation. Indeed, Th1 cells cultured with inhibitor continued to express an increased amount of IL-10 after a subsequent round of restimulation with the inhibitors removed from the culture (FIG. 5 A). This led us to investigate the epigenetic status of the IL-10 promoter since epigenetic changes are able to elicit a prolonged change in gene expression. ChIP analysis of the IL-10 promoter using four primer sets spanning the promoter (FIG. 5 B) revealed a GSK3 inhibitor-induced increase in histone H3 acetylation, a marker for active gene transcription (FIG. 5 C). H3K4 trimethylation and H3K27 methylation were also examined using the same promoter-spanning primer sets and additionally a primer pair situated in the coding region of IL-10 (IL-10C2; see FIG. 5B). H3K4 trimethylation is generally regarded as a marker for transcriptionally active genes and H3K27 methylation as a mark of repression (REFS). GSK3 inhibitor treatment of Th1 cells resulted in an increase in H3K4 trimethylation and a decrease in H3K27 trimethylation (FIGS. 5 D and E).

### Example 7

### Exogenous IL-10 is not Required for GSK3 Inhibitor Induced IL-10 Production in Th2 Cells

Splenocytes from a naive Tg4 mouse were cultured in the presence of peptide MBP Ac1-9 (10 μg/ml), anti IFNλ and IL-4 with IL-2 added on day 3 of culture to polarise CD4+ cells towards a Th2 phenotype. On day 7 of culture, Th2 cells were restimulated with fresh irradiated APCs from either a B10_PL or B10_PL IL10^{-/-} mouse in the presence of DMSO (1:1000; Control) or CHIR99021 (2 μM). IL-2 was added on day 3 of culture and intracellular cytokine staining for IL-4 and IL-10 carried out on day 7 of restimulation after PMA and lonomycin stimulation. The results are shown in FIG. 7A.

Tissue culture supernatants were taken on day 3 of restimulation and analysed for IL-10 by ELISA. The results are shown in FIG. 7B.

### Example 8

### GSK3 Inhibitor Treatment Increases IL-10 Secretion In Vivo

Mice (three per group) underwent two intranasal treatments with peptide Ac[1-9]MBP. Two hours prior to a third treatment with peptide, mice were injected with either 0.6 mg/kg SB216763 or vehicle control. Three days after this third peptide treatment the spleens were taken and splenocytes cultured for five days with peptide Ac[1-9]MBP and IL-2 before intracellular cytokine staining for IFNλ and IL-10 was carried out after PMA and lonomycin stimulation. The results are shown in FIG. 8.

All publications mentioned in the above specification are herein incorporated by reference. Various modifications and variations of the described methods and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention which are obvious to those skilled in antigen-specific immunotherapy or related fields are intended to be within the scope of the following claims.

1. A composition which comprises a tolerogenic peptide and a glycogen synthase kinase-3 (GSK-3) inhibitor.

2. A composition according to claim 1, wherein the tolerogenic peptide is a peptide from a self-antigen or an allergen.

3. A composition according to claim 1 or 2, wherein the GSK-3 inhibitor is a lithium ion

4. A composition according to claim 1 or 2 wherein the GSK-3 inhibitor is CHIR99021, SB216763 or SB627772.

5. A composition according to any preceding claim for use in method for treating and/or preventing an autoimmune disease, an allergic reaction, a condition associated with transplant rejection, or a condition in which inflammation and CD4+ T cells contribute to pathogenesis such as cerebral malaria, atherosclerosis, type II diabetes and neuropathic pain.

6. A kit comprising a tolerogenic peptide and a GSK-3 inhibitor for separate, simultaneous or sequential administration.

7. A method for treating and/or preventing a disease associated with pro-inflammatory T cells which comprises the step of administering a tolerogenic peptide and a GSK-3 inhibitor to the subject.

8. A method according to claim 7, wherein the tolerogenic peptide and GSK-3 inhibitor are administered simultaneously, sequentially or separately.

9. A method according to claim 7, which comprises two treatment stages:
(i) an initial treatment stage with both peptide and GSK-3 inhibitor; and
(ii) a subsequent treatment phase with peptide in the absence of GSK-3 inhibitor

10. A method according to any of claims 7 to 9, wherein the disease associated with proinflammatory T cells is selected from: an autoimmune disease, an allergic reaction, a condition associated with transplant rejection, and a condition in which inflammation and CD4+ T cells contribute to pathogenesis such as cerebral malaria, atherosclerosis, type II diabetes and neuropathic pain.

11. A method according to any of claims 7 to 10, wherein the subject has a pre-existing condition, or is about to undergo or undergoing transplantation.

12. A method according to claim 10, wherein the subject has a pre-existing Th1/Th17 or Th2 immune response specific for a self-antigen or allergen.

13. A GSK-3 inhibitor for use in enhancing antigen-specific immunotherapy.

14. A GSK-3 inhibitor for use in accelerating the peptide-mediated shift in secretion profile of T cells from pro-inflammatory to anti-inflammatory cytokines.

15. A composition according to any of claims 1 to 5 or a kit according to claim 6 for use in treating and/or preventing a disease associated with pro-inflammatory T cells.

16. A composition or kit according to claim 15, wherein the disease associated with proinflammatory T cells is selected from: an autoimmune disease, an allergic reaction, a condition associated with transplant rejection, and a condition in which inflammation and CD4+ T cells contribute to pathogenesis such as cerebral malaria, atherosclerosis, type II diabetes and neuropathic pain.
